(19) United States
(12) Patent Application Publication
(10) Pub. No.: US 2026/0155259 A1
(43) Pub. Date: Jun. 4, 2026

(54) METHODS AND DEVICES FOR DETERMINING A RISK OF DEVELOPING SEPSIS FOR A PATIENT

(71) Applicant: Beckman Coulter, Inc., Brea, CA (US)

(72) Inventors: Trinity Crapse, Brea, CA (US); Daniel Nichita, Brea, CA (US)

(21) Appl. No.: 19/148,659

(22) PCT Filed: Jan. 23, 2024
(86) PCT No.: PCT/US2024/012503

§ 371 Date: Jul. 16, 2025

Related U.S. Application Data
(60) Provisional application No. 63/441,024, filed on Jan. 25, 2023.
(60) Provisional application No. 63/465,345, filed on May 10, 2023.

(51) Int. Cl. A61B 5/00

(52) U.S. Cl. CPC G16H 50/30; G16H 10/40; G16H 10/60

(57) ABSTRACT

A computer-implemented method of determining, for a patient, a risk of developing sepsis is described. The method comprises obtaining, at a computing device, a set of patient data indicative of at least one of: a patient age value, a patient heart rate value, a patient respiratory rate value, a patient diastolic blood pressure value, a patient systolic blood pressure value, a patient temperature value, and a patient oxygen saturation value; and at least one value of a Monocyte Distribution Width (MDW) of the patient. Further, the method comprises determining, based on processing the obtained set of patient data with the computing device, at least one sepsis indicative of the risk for a sepsis event occurring at the patient.

### CROSS-REFERENCE TO RELATED APPLICATIONS

This application claims the benefit of priority to U.S. Provisional Patent Application Ser. No. 63/441,024 filed Jan. 25, 2023, and of U.S. Provisional Patent Application Ser. No. 63/465,345 filed May 10, 2023, the disclosures of which are incorporated herein in its entirety by reference.

### TECHNICAL FIELD

The present disclosure generally relates to the field of clinical decision support. In particular, the present disclosure relates to a computer-implemented method of determining, for a patient, subject or individual, a risk of developing sepsis. The present disclosure also relates to a computing device or system configured to carry out steps of such method, to a corresponding computer program instructing the computing device or system to perform steps of such method, and to a computer-readable medium storing such computer program.

### BACKGROUND

Sepsis can be a life-threatening organ dysfunction caused by a dysregulated host response to infection. In particular, according to the present disclosure. “Sepsis” may indicate severe sepsis or septic shock by Sepsis-2 criteria, according to Levy et. Al 2001 Sepsis Definitions Conference guidelines (Crit Care Med 2003 Vol. 31, No. 4), which is incorporated herein by reference.

The key to survival and the avoidance of serious complications is generally the early diagnosis and treatment of sepsis. However, this can be difficult and challenging even for experienced clinicians to achieve because initial symptoms are often subtle and non-specific. Therefore, sepsis detection and antibiotic treatment can in practice often be delayed up to by several hours and may thus contribute to overall mortality recorded at clinics and hospitals. Although various clinical risk scores like the Systemic Inflammatory Response Syndrome (SIRS), the quick Sequential Organ Failure Assessment (qSOFA) and the Modified Early Warning Assessment (MEWS) scores are often employed for sepsis screening, these simple rule-based scores typically have poor overall performance for early sepsis detection. Most studies of these scores report on their ability to predict in-hospital mortality or other adverse events like Intensive Care Unit (ICU) admission in patients with infection rather than predict sepsis per se. Although results vary widely among studies, in general, SIRS has been found to be sensitive for predicting adverse outcomes but not specific, whereas the opposite has been found of qSOFA.

### SUMMARY OF THE INVENTION

Given the limited performance of the aforementioned traditional methods of assessing the risk of developing sepsis, like the scoring methods based on SIRS, qSOFA, and MEWS score, it is desirable to provide for sepsis detection. In particular, it may be desirable to allow for a computer-implemented early sepsis detection to facilitate early intervention and treatment of patients when this can be most beneficial and lead to the best clinical outcomes.

This is achieved by the subject matter of the independent claims, wherein further exemplary embodiments are included in the dependent claims and the following description.

A first aspect of the present disclosure refers to a computer-implemented method of determining, for a patient, a risk of developing sepsis. A second aspect of the present disclosure refers to a computing device including one or more processors for data processing, wherein the computing device is configured to carry out steps of the method according to the first aspect of the present invention. A third aspect of the present disclosure refers to a computer program, which, when executed by one or more processors of a computing device, instructs the computing device to perform steps of the method according to the first aspect of the present invention. A fourth aspect of the present disclosure refers to a non-transitory computer-readable medium storing a computer program according to the third aspect of the present invention. Any disclosure presented herein with reference to one aspect of the present disclosure equally applies to any other aspect of the present disclosure, unless explicitly stated otherwise.

In particular, the first aspect, the second aspect, the third aspect and the fourth aspect of the present disclosure can assist healthcare providers, such as clinicians or medical doctors, with the detection of sepsis. In particular, aspects of the present disclosure can aid healthcare providers (HCPs) in early detection of sepsis, for example with adult patients at risk of sepsis in an Emergency Department (ED) and in patient settings.

According to a first aspect of the present disclosure, there is provided a computer-implemented method of determining, for a patient subject or individual, a risk of developing sepsis. The method comprises:
obtaining, at a computing device, a set of patient data indicative of:
   at least one of: a patient age value, a patient heart rate value, a patient respiratory rate value, a patient diastolic blood pressure value, a patient systolic blood pressure value, a patient temperature value, and a patient oxygen saturation value; and
   at least one value related to a Monocyte Distribution Width (MDW) of the patient; and
determining, with the computing device, based on the obtained set of patient data, at least one sepsis score indicative of the risk for a sepsis event occurring at the patient.

Alternatively, the method according to the first aspect comprises:
obtaining. at a computing device, a set of patient data indicative of:
   a patient age value, a patient heart rate value, a patient respiratory rate value, a patient diastolic blood pressure value, a patient systolic blood pressure value, a patient temperature value, and a patient oxygen saturation value; and
   optionally at least one value related to a Monocyte Distribution Width (MDW) of the patient; and
determining, with the computing device, based on the obtained set of patient data, at least one sepsis score indicative of the risk for a sepsis event occurring at the patient.

Determining the sepsis score in accordance with the method of the first aspect can allow for an accurate detection of sepsis, in particular an early detection of sepsis at the patient. Hence, the method and device described herein can assist HCPs with the early identification of patients, for instance adult patients, at risk for sepsis in emergency department (ED) and in patient care settings.

The inventors surprisingly found that taking one or more of, for example all of, a patient age value, a patient heart rate value, a patient respiratory rate value, a patient diastolic blood pressure value, a patient systolic blood pressure value, a patient temperature value, a patient oxygen saturation value of or associated with the patient into consideration, the risk for the patient suffering from or developing sepsis can be more accurately and reliably determined, for example when compared to an assessment based on SIRS or other scoring methods mentioned above. The inventors also found that taking at least one value related to and/or associated with MDW into account, the accuracy in the determination of the risk for sepsis, and hence the sepsis score, can be further improved. Accordingly, by means of the computer-implemented method described herein, risk stratification, for example at hospitals or emergency departments, can be significantly improved, as the method and device described herein may allow to efficiently, reliably, and/or accurately risk stratify a patient, for example rule in or rule out a patient as a sepsis patient, as being at risk for sepsis and/or as having a particular risk for developing sepsis. For instance, the method and device described herein allow for stratifying a patient as having a high, medium or low risk for sepsis. Herein, risk level is also referred to as “risk tier”.

Further, determining the sepsis score can advantageously allow for a quantitative assignment of a health risk or risk status to the patient, such as for example a low, medium or high risk for sepsis. In particular, a number of false positives and/or a number of false negatives in determining or assessing whether a patient has a low, medium or high risk for sepsis, such as a risk below, between or above a threshold risk, can be advantageously reduced by means of the computer-implemented method described herein.

Moreover, by determining the risk for sepsis and/or the sepsis score based on, for example at least one value of MDW and at least one of a patient age value, a patient heart rate value, a patient respiratory rate value, a patient diastolic blood pressure value, a patient systolic blood pressure value, a patient temperature value and a patient oxygen saturation value, a health state of the patient can be comprehensively examined or evaluated. As a consequence, the determination of the risk for sepsis can be individualized to individual patients or patient sub-groups, which can further reduce the number of false positives and/or false negatives. Also, the time required for determining the risk of sepsis can be significantly reduced. Herein the sepsis score may also referred to as: “sepsis risk score”.

The present disclosure, therefore, can provide for an improved clinical decision support system, for example allowing to efficiently, reliably and/or accurately risk stratify a patient as, for example, having a high, medium or low risk for sepsis. Stratification into more or less than three risk levels or tiers is envisaged in the context of the present disclosure.

For instance, aspects of the present disclosure may facilitate earlier discharge of patients from hospitals for patients having a low sepsis risk or score or being not at risk for sepsis, and earlier intervention for those patients who are more likely to experience a sepsis event or are at risk for sepsis or have sepsis. Hence, unnecessary hospitalization and anxiety among patients may be efficiently avoided or reduced. Also, certain procedures and protocols can be avoided at hospitals, which can result in a better patient experience, improved utilization of healthcare resources and cost savings, all while maintaining a high safety profile.

For instance, the method and device described herein can allow to reduce or avoid overcrowding at emergency departments. Also, the use of scarce healthcare resources, such as observation beds, and diagnostic modalities, such as cardiac stress tests, angiograms and imaging studies, can be optimized. Further, the patient experience can be improved, for example by reducing the length of an emergency department stay. Alternatively or additionally, the documentation of risk assessment can be automated and the ability for shared decision-making with patients about diagnostic and treatment choices can be significantly improved.

According to the present disclosure a patient parameter (herein also referred to as: “parameter”) may be any vital sign or any laboratory result on which the determination of the at least one sepsis score is based. For instance, a patient parameter may be patient age, patient heart rate, patient respiratory rate, patient diastolic blood pressure, patient systolic blood pressure, patient temperature, patient oxygen saturation, patient WBC or patient MDW. Any disclosure presented herein with reference to a patient parameter may apply to any patient parameter, e.g. to one or more of patient age, patient heart rate, patient respiratory rate, patient diastolic blood pressure, patient systolic blood pressure, patient temperature, patient oxygen saturation, patient WBC, patient MDW.

As used herein, any parameter value of the patient or associated with the patient, for example any parameter value contained in the set of patient data, such as the at least one value of MDW, the patient heart rate value, the patient respiratory rate value, the patient diastolic blood pressure value, the patient systolic blood pressure value, the patient temperature value, the patient oxygen saturation value, and/or the patient WBC value, can be obtained based on a measurement, for example based on or using one or more samples from the patient, such as blood samples. This applies to any clinical data or patient data, such as values of vital signs and/or laboratory results of a patient, described herein as being usable to compute the sepsis score.

The parameters patient heart rate, patient respiratory rate, patient diastolic blood pressure, patient systolic blood pressure, patient temperature, and patient oxygen saturation are generally referred to herein as vital signs or vital parameters of or associated with the patient.

According to an embodiment, the set of patient data is indicative of at least one of a patient age value, a patient heart rate value, a patient respiratory rate value, a patient diastolic blood pressure value, a patient systolic blood pressure value, a patient temperature value, a patient oxygen saturation value, and a patient White Blood cell Count (WBC) value.

Optionally the set of patient data (also referred to as patient data) may include time information for at least some of the parameter values included or indicated by the set of patient data. In particular, any one or more of the at least one value of MDW, the patient heart rate value, the patient respiratory rate value, the patient diastolic blood pressure value, the patient systolic blood pressure value, the patient temperature value, the patient oxygen saturation value, and/or optionally the patient WBC value, can be associated with or include a time information, such as for example a timestamp. The same applies to any other parameter value described herein as being potentially used for the computation of the sepsis risk score. The timestamp or time information associated with the one or more parameter values may indicate one or more of: (i) a measurement time. i.e., a time at which the respective parameter value has been measured at the patient. (ii) a time of recordation of the parameter value, and (iii) a time of receipt of the parameter value at the computing device.

As used herein, determining the sepsis risk or sepsis score may include computing and/or assessing the risk for sepsis. For instance, determining may generally relate to or include finding out or coming to a decision about the risk for sepsis. In particular, determining the sepsis risk or sepsis score may optionally include a corresponding reasoning or assessment of the risk, a calculation and/or a computation of the risk or sepsis score. Alternatively or additionally, computing the risk for sepsis may include determining based on mathematical means, algorithms and/or calculations, the risk for sepsis. In particular the calculations may be computer-aided, computer-assisted and/or computer-implemented. For example, determining the sepsis risk or sepsis score may be carried out by using a determination algorithm. In particular, the determination algorithm can be configured to receive and process input data, the input data comprising and/or being based on the set of patient data. The determination algorithm can be configured to generate output data, the output data being indicative of the sepsis risk or the sepsis score. Alternatively or additionally, assessing the sepsis risk may include determining an importance, a significance, a value, a level and/or a tier for the risk for sepsis and/or the sepsis score.

As used herein, obtaining the set of patient data may comprise accessing said set data and/or retrieving the set of patient data e.g. from the at least one memory of the computing device that carries out the method of the present disclosure, from the memory of another computing device, or from another remote data storage (a database, a secondary memory, a cloud storage or the like). Accordingly, in some cases, retrieving the set of patient data may comprise downloading said set. Additionally or alternatively, obtaining the set of patient data may comprise receiving said set. e.g. from a user or a computing device different from the computing device accessing the data. The two options are not mutually exclusive. For instance, obtaining the set of patient data may comprise receiving said set data, storing said set in the memory of the computer device and retrieving the set of patient data by accessing said memory.

Further, the sepsis score may be indicative, descriptive or reflective of the likelihood or probability for sepsis to occur in the patient or of the likelihood or probability for the patient having sepsis. In particular, the sepsis score may refer to a numerical measure indicative of the determined risk, likelihood or probability for sepsis to occur at the patient, for example within a predetermined period of time from a time of receipt of at least a subset of the set of patient data and/or a time of computing the sepsis score. The predetermined period of time may be comprised between 1 hour and 10 hours, for example between 2 hours and 5 hours. Preferably the predetermined period of time may be equal to about 3 hours. Accordingly, determining the risk for sepsis to occur at the patient may refer to determining a likelihood or probability for an onset of sepsis to fall within or occur within a predetermined period of time from an actual computation of the sepsis score by the computing device and/or from receipt of the set of patient data at the computing device. The predetermined period of time is also referred to herein as “predictive time window”.

In a non-limiting example, the sepsis score may be provided on an arbitrary scale ranging from a minimum value, for example zero or 0, to a maximum value, for example one or 100. Any other scale, including relative and absolute scales, can be used to represent the sepsis score. Alternatively or additionally, the sepsis score may indicate a risk level or risk tier, such as low risk, medium risk, high risk.

The method according to the present disclosure can comprise determining at least one sepsis score indicative of the risk for a sepsis event or indicative of the risk of sepsis occurring at the patient. In particular, according to an embodiment of the method according to the present invention, determining the at least one sepsis score includes computing the risk for sepsis occurring at the patient based on, e.g. by using, at least one machine learning (ML) model, e.g. a predictive ML model. For example, the ML model can be configured to compute a numeric sepsis risk score by using patient demographic (such as age or gender) and patient clinical data. Patient clinical data may include values of one or more patient vital sign and/or laboratory results obtained from the EHR. In particular, patient heart rate value, a patient respiratory rate value, patient diastolic blood pressure, patient systolic blood pressure, patient temperature, and patient oxygen saturation are patient vital signs. For instance, a patient WBC value and patient MDW value are laboratory results obtained from the EHR.

In an example, the machine learning model implemented at the computing device and optionally used to compute or determine the sepsis score can be configured to receive and process input data, the input data comprising and/or being based on the set of patient data. For instance, the determination algorithm mentioned above may comprise or consist of the ML model.

The at least one machine learning model can, for example, comprise a trained gradient boosting algorithm, a trained artificial neural network, a trained feed forward neural network, a trained convolutional neural network and/or a trained deep neural network. Training of the ML model will be described in more detail hereinbelow.

In particular, some aspects of the present disclosure can use a predictive machine learning (ML) algorithm to analyze relevant clinical data or patient data such as values of one or more patient vital signs and/or one or more laboratory results of a patient, to compute a sepsis risk score. Optionally, some aspects of the present disclosure can provide information indicative of one or more key parameters contributing to the sepsis score (also referred to as risk score or sepsis risk score), and potentially other relevant clinical information to further aid HCPs in their interpretation of the risk score and its use in the decision-making process.

According to an embodiment, the set of patient data is indicative of at least one of: a plurality of patient heart rate values, a plurality of patient respiratory rate values, a plurality of patient diastolic blood pressure values, a plurality of MDW values, a plurality of patient systolic blood pressure values, a plurality of patient temperature values, a plurality of patient oxygen saturation values, and a plurality of patient WBC values.

According to the present disclosure, the plurality of values of a patient parameter may be obtained at the same or different time points. In particular, values of a plurality of values of a patient parameter, such as a plurality of heart rate values, are obtained at different time points, thereby allowing, for instance, for taking into account the variation of the parameter over time and the effect and/or impact of said variation on the computed sepsis score. For instance, each value of a plurality of values of a patient parameter is obtained at respective time points, in such a way that, for each pair of values of said plurality of values, the time point at which the first value of that pair of values is obtained, is different from the time point at which the second value of that pair of values is obtained.

According to the present disclosure, corresponding time information for each value of the plurality of values of a patient parameter may be included in the set of patient data, for example in the form of a timestamp associated with the respective value. The timestamps or time information of different values of said plurality of values may be identical, for example when two measurements of a parameter have been performed at the same time, or may differ, for example when two consecutive measurements of a parameter have been made.

In an exemplary embodiment, the set of patient data is indicative of the plurality of patient heart rate values and determining the at least one sepsis score is based on a maximum value of the patient heart rate, a minimum value of the patient hearth rate, a most recent value of the patient heart rate and/or one or more mean values of the patient heart rate. Alternatively or additionally, the set of patient data can be indicative of the plurality of patient respiratory rate values and determining the at least one sepsis score is based on a maximum value of the patient respiratory rate, a minimum value of the patient respiratory rate, a most recent value of the patient respiratory rate and/or one or more mean values of the patient respiratory rate. Alternatively or additionally, the set of patient data can be indicative of the plurality of MDW values and determining the at least one sepsis score is based on a maximum value of MDW, a minimum value of MDW, a most recent value of MDW and/or one or more mean values of the MDW. Alternatively or additionally, the set of patient data can be indicative of the plurality of patient diastolic blood pressure values and determining the at least one sepsis score is based on a maximum value of the patient diastolic blood pressure, a minimum value of the patient diastolic blood pressure, a most recent value of the patient diastolic blood pressure and/or one or more mean values of the patient diastolic blood pressure. Alternatively or additionally, the set of patient data can be indicative of the plurality of patient systolic blood pressure values and determining the at least one sepsis score is based on a maximum value of the patient systolic blood pressure, a minimum value of the patient systolic blood pressure, a most recent value of the patient systolic blood pressure and/or one or more mean values of the patient systolic blood pressure. Alternatively or additionally, the set of patient data can be indicative of the plurality of patient oxygen saturation values and determining the at least one sepsis score is based on a maximum value of the patient oxygen saturation, a minimum value of the patient oxygen saturation, a most recent value of the patient oxygen saturation and/or one or more mean values of the patient oxygen saturation. Alternatively or additionally, the set of patient data can be indicative of the plurality of patient WBC values and determining the at least one sepsis score is based on a maximum value of the patient WBC, a minimum value of the patient WBC, a most recent value of the patient WBC, and/or one or more mean values of the patient WBC. Alternatively or additionally, the set of patient data is indicative of the plurality of patient temperature values and determining the at least one sepsis score can be based on a maximum value of the patient temperature, a minimum value of the patient temperature, a most recent value of the patient temperature and/or one or more mean values of the patient temperature.

Accordingly, any of the maximum parameter value(s), the minimum parameter value(s), the most recent value(s) and/or the mean value(s) can be computed by the computing device to determine the sepsis score. Again, this can allow to take into account variation over time, the evolution the development of one or more of the aforementioned patient parameters for determining the sepsis score. Also, the sepsis score may be updated upon receiving one or more further parameter values of one or more parameters described herein as being usable for determining the sepsis score.

One or more of the aforementioned parameter values may be computed, determined and/or selected by the computing device based on the obtained or received set of patient data. Accordingly, the method may comprise determining, computing and/or selecting a minimum value, a maximum value, a most recent value and/or a mean value among a plurality of parameter values of a particular parameter contained in the set of patient data, such as for example among the plurality of patient heart rate values, among the plurality of patient respiratory rate values, among the plurality of patient diastolic blood pressure values, among the plurality of MDW values, among the plurality of patient systolic blood pressure values, among the plurality of patient temperature values, among the plurality of patient oxygen saturation values, and/or among the plurality of patient WBC values. The same applies to any other parameter described herein as being potentially usable for computing the sepsis score. Alternatively or additionally, one or more of these parameter values, i.e., the minimum value, the maximum value, the most recent value and/or the mean value of a plurality of parameter values of a particular parameter, may be received as patient data at the computing device and used as input data to compute the sepsis score.

In an exemplary embodiment, the input data comprise the maximum value of the patient heart rate the minimum value of the patient hearth rate, the most recent value of the patient hearth rate and/or the one or more mean values of the patient heart rate. Alternatively or additionally, the input data can comprise the maximum value of the patient respiratory rate, the minimum value of the patient respiratory rate, the most recent value of the patient respiratory rate and/or the one or more mean values of the patient respiratory rate. Alternatively or additionally, the input data can comprise the maximum value of the patient diastolic blood pressure, the minimum value of the patient diastolic blood pressure, the most recent value of the patient diastolic blood pressure and/or the one or more mean values of the patient diastolic blood pressure. Alternatively or additionally, the input data can comprise the maximum value of the patient systolic blood pressure, the minimum value of the patient systolic blood pressure the most recent value of the patient systolic blood pressure and/or the one or more mean values of the patient systolic blood pressure. Alternatively or additionally, the input data can comprise the maximum value of the patient oxygen saturation, the minimum value of the patient oxygen saturation, the most recent value of the patient oxygen saturation and/or the one or more mean values of the patient oxygen saturation. Alternatively or additionally, the input data can comprise the maximum value of the patient WBC, the minimum value of the patient WBC, the most recent value of the patient WBC and/or the one or more mean values of the patient WBC. Alternatively or additionally, the input data can comprise the maximum value of the patient temperature, the minimum value of the patient temperature, the most recent value of the patient temperature and/or the one or more mean values of the patient temperature.

In an embodiment, the set of patient data is indicative of a plurality of MDW values of the patient, for example obtained at different times or time points, and determining the at least one sepsis score is based on a maximum value of the MDW of the patient, a minimum value of the MDW of the patient, a most recent value of the MDW of the patient and/or one or more mean values of the MDW of the patient. This may allow to further improve risk stratification.

In an exemplary embodiment, the sepsis score or risk score can be recomputed every time one or more new; updated and/or additional parameter values of patient data are received at the computing device, for example detected in the EHR of the respective patient. Optionally, the sepsis score or risk score can be provided to and/or stored in the EHR where HCPs can view the score during their routine EHR activities while managing patients.

In an embodiment, the method further comprises determining, based on the at least one sepsis score, a sepsis risk level or risk tier. In particular, the sepsis risk level comprises selecting the sepsis risk level or risk tier from a plurality of risk levels nor risk tiers. Selecting a risk level or tier may include determining a risk level or tier that matches, corresponds to or is associated with the determined sepsis score. Determining the sepsis risk level or tier may further improve risk stratification based on the computed sepsis score and may simplify interpretation thereof, for example by a clinician.

For instance, selecting the sepsis risk level from the plurality of risk levels may comprises comparing the at least one sepsis score with one or more risk score thresholds.

In an example, the plurality of risk levels can comprise a first risk level, a second risk level and a third risk level, wherein if the at least one sepsis score is lower than a first threshold, the first risk level is selected as sepsis risk level, if the at least one sepsis score is greater than the first threshold and lower than a second threshold, the second risk level is selected as sepsis risk level, and if the at least one sepsis score is greater than the second threshold, the third risk level is selected as sepsis risk level.

The sepsis risk score can also be used to trigger active notifications such as EHR popup alerts. To facilitate interpretation, the risk score or sepsis score can be stratified into a plurality of risk levels or tiers, for example three risk tiers low, medium, and high risk. Herein risk tiers can also referred to as “risk levels”. Alternatively more than three or only two risk tiers can be used.

Alternatively, selection and/or determination of the risk level may be carried out by using an ML model which is trained to select and/or determine the risk level by using the input data. In this case, the risk level may be provided directly as output of the ML data. Accordingly, an output of the computing device may include an indication or information indicative of the risk level or risk tier, to which the patient is associated, according to or based on the determined sepsis score.

In another embodiment, the set of patient data comprises, e.g. consists of, the patient age value, the patient heart rate value, the patient respiratory rate value, the patient diastolic blood pressure value, the patient systolic blood pressure value, the patient temperature value, the patient oxygen saturation value and optionally the patient WBC value. Using all these parameters and optionally WBC may further improve accuracy of the determined sepsis score. In particular a plurality of parameter values for each of the aforementioned patient parameters. i.e., of the patient age, the patient heart rate, the patient respiratory rate, the patient diastolic blood pressure, the patient systolic blood pressure, the patient temperature, the patient oxygen saturation and optionally the patient WBC, may be included in the set of patient data, as described above.

In another embodiment, the set of patient data comprises, e.g. consists of, the patient heart rate value, the patient respiratory rate value, the patient diastolic blood pressure value, the patient systolic blood pressure value, the patient temperature value and the patient oxygen saturation value. Using all these parameters may further improve accuracy of the determined sepsis score. In particular, a plurality of parameter values for each of the aforementioned patient parameters. i.e., the patient heart rate, the patient respiratory rate, the patient diastolic blood pressure, the patient systolic blood pressure, the patient temperature and the patient oxygen saturation may be included in the set of patient data, as described above.

According to an embodiment, the method according to the present invention comprises obtaining and/or determining contextual information associated with the determined sepsis score and/or sepsis risk level. For example, the contextual information may include information or data indicative or reflective of a computational basis leading to the determination of the determined sepsis score and/or sepsis risk level.

The contextual information may include data or information for interpreting the determined sepsis score and/or sepsis risk level. Exemplary contextual information may include one or more notifications, messages or other informative means, which may optionally be provided at a user interface of the computing device. In an example, the contextual information may include an indication or information about the parameters used for computing the determined sepsis score and/or sepsis risk level. Exemplary contributing parameters may include the patient age, a patient heart rate value, a patient respiratory rate value, a patient diastolic blood pressure value, a patient systolic blood pressure value, a patient temperature value, a patient oxygen saturation value, optionally a patient WBC value and/or a MDW value of the patient. In particular, the method comprises providing, e.g. displaying, to the user an explanation of the top critical contributors to the sepsis risk score computation.

In particular, the method according to the present invention comprises receiving patient data from the EHR as soon as patients are registered in the EHR and their vital signs and other relevant tests are entered into the system, an action typically first performed during patient triage on ED/Inpatient admission. As additional vitals and other laboratory tests such as CBCs are performed during the patient workup, the method comprises receiving and analyze this data and redetermining, e.g. recomputing the sepsis risk score. For example, every time a new risk score is computed by the model, the method comprises delivering the score back to the EHR where it may be viewed by HCPs.

According to an embodiment, the method of the present invention is a method for determining, for a patient of a particular or threshold age, for example for a patient of 18 years of age or older, a risk of developing sepsis.

According to an embodiment of the method of the present invention, determining the sepsis score includes computing the risk for the sepsis event occurring at the patient based on at least one ML model. The ML model is configured to receive and process input data. According to one embodiment, the ML model is configured and trained to assess the sepsis risk in (adult) patients using input data comprising information like patient demographics, patient vitals and other lab test results of the patient. In particular, an adult patient is a patient of 18 years of age or older.

According to an embodiment, the method further comprises monitoring an electronic health record (EHR) associated with the patient and/or monitoring the patient data or set of patient data, for example stored on a data storage of the computing device or accessed by the computing device. For instance, the method of the present disclosure may comprise re-computing and/or updating, by the computer device, the sepsis score upon receipt of one or more parameter values associated with the patient by using the at least one ML model. Accordingly, the sepsis score may be updated and/or re-computed whenever one or more new, additional and/or updated parameter values for the patient data become available or are obtained at the computing device, for example in the form of patient data.

In particular, the method can comprise determining the sepsis risk score by using the ML model, current patient data and past patient data obtained within or over a time window in the past (also referred to as “trending window” or predetermined period of time), as will be further described hereinbelow with reference to FIG. 3.

For instance, the method may comprise determining all parameter values falling within the trending window. This may include determining whether the timestamp of a parameter value is in the trending window. Optionally, determining the at least one sepsis score comprises:
computing one or more of the minimum value, the maximum value, the most recent value and/or a mean value of a subset of values of a plurality of parameter values of a patient parameter, each value of the subset of values being obtained at a time point comprised within the trending window; and
using said one or more of the minimum value, the maximum value, the most recent value and/or a mean value used to compute the sepsis score. e.g. as described hereinabove.

In particular, at a given time point, the computing device and/or at least one ML model may use all the values of all patient parameters, for instance of the vital signs. WBC and MDW, recorded in the past during the trending window to compute the sepsis score, determine the risk for sepsis and/or compute the probability of a sepsis onset. For example, the ML model may be configured to consider patient data over a period of up to 24 hours, preferably up to 12 hours in the past (relative to a time of computation of the sepsis score) and to predict the sepsis risk or sepsis onset for a predetermined period of time in the future (ahead of the time of computation of the sepsis score), such as for a predictive window of 3 hours in advance, as described above. Optionally, the computing device and/or ML model may stop computing the sepsis risk score where the patient is discharged from the hospital, or when the patient is transferred to the ICU, operating room or post-op recovery ward.

According to some embodiments, the input data for the at least one ML model comprise or are based on a set of patient data. In particular, the set of patient data is indicative of at least one of:
1. At least one Patient age value;
2. At least one patient Heart rate value;
3. At least one Respiratory rate value;
4. At least one Diastolic blood pressure value;
5. At least one Systolic blood pressure value;
6. At least one Temperature value; and
7. At least one Oxygen saturation value.

Accordingly, the sepsis score and/or the risk for sepsis may be determined based on one or more of, in particular all of, the aforementioned parameters or parameter values. Optionally, the set of patient data is further indicative of at least one WBC value.

According to some embodiments, the input data for the at least one ML model comprise or are based on a set of patient data. In particular, the set of patient data is indicative of at least one of:
1. At least one Patient age value;
2. At least one patient Heart rate value;
3. At least one Respiratory rate value;
4. At least one Diastolic blood pressure value;
5. At least one Systolic blood pressure value;
6. At least one Temperature value;
7. At least one Oxygen saturation value; and
8. At least one WBC value.

Accordingly, the sepsis score and/or the risk for sepsis may be determined based on one or more of, in particular all of, the aforementioned parameters or parameter values. Also a plurality of one or more of the aforementioned parameter values may be comprised in the set of patient and used to determine the at least one sepsis score.

According to some embodiments, the input data for the at least one ML model comprise or are based on a set of patient data. In particular, the set of patient data is indicative of at least one of:
1. At least one Patient age value;
2. At least one patient Heart rate value;
3. At least one Respiratory rate value;
4. At least one Diastolic blood pressure value;
5. At least one Systolic blood pressure value;
6. At least one Temperature value;
7. At least one Oxygen saturation value;
8. At least one WBC value; and
9. At least one MDW value.

Accordingly, the sepsis score and/or the risk for sepsis may be determined based on one or more of, in particular all of, the aforementioned parameters or parameter values. Also a plurality of one or more of the aforementioned parameter values may be comprised in the set of patient and used to determine the at least one sepsis score.

An MDW value or “a value related to MDW” in the context or the present disclosure can refer to a parameter indicative of the monocyte cell population of the patient's blood and/or containing information about or related to MDW. MDW or the MDW value may be indicative of a variation or dispersion of the monocyte cell size within the monocyte cell population of the patient's blood. Such variation or dispersion in the size of monocyte blood cells can be obtained by various measurements, including measurements based on the Coulter principle, flowcytometry, fluorescence, light scattering measurements and others.

For instance, when CBC is obtained from a blood sample, using an analyzer such as a hematology analyzer on the same blood sample may further provide data about a subpopulation of cells that is much richer than simply a count or proportion of those cells compared to other subpopulations of cells within a sample. For example, a monocyte cell population parameter that reflects monocyte activation may be obtained. One such monocyte parameter is monocyte distribution width (referred to herein as MDW, and also referred to as monocyte anisocytosis). MDW represents the volume distribution of the monocyte population in a blood sample; thus, this morphometric parameter reflects variability in monocyte cell volume.

Morphological changes in monocyte cell volume can occur early as a result of pathogen recognition-induced monocyte activation, and thus MDW can be altered early in disease trajectory. MDW has demonstrated capability in identification of patients with sepsis in high-risk populations (Crouser et al. Crit Care Med. 2019; 47(8): 1018-1025; Crouser et al. Chest. 2017; 152(3):518-526; Crouser et al. Intensive Care; 2020; 8:33).

In particular, MDW can be regarded a morphometric leukocyte biomarker that can signify the standard deviation in the width distribution of monocytes, a leukocyte critical to the initiation of the innate immune response, and an early indicator of infection.

In some embodiments, the set of patient data is indicative of at least one Patient age value, at least one patient Heart rate value, at least one Respiratory rate value, at least one Diastolic blood pressure value, at least one Systolic blood pressure value, at least one Temperature value, at least one Oxygen saturation value, at least one WBC value, and at least one MDW value.

According to some embodiments, the input data for the at least one ML model comprise or are based on a set of patient data. In particular, the set of patient data is indicative of one or more of:
1. At least one Patient age value;
2. At least one patient Heart rate value;
3. At least one Respiratory rate value;
4. At least one Diastolic blood pressure value;
5. At least one Systolic blood pressure value;
6. At least one Temperature value;
7. At least one Oxygen saturation value;
8. At least one WBC value;
9. At least one MDW value;
10. At least one lactate value;
11. At least one creatinine value,
12. At least one lymphocyte value,
13. At least one platelet value;
14. At least one neutrophil value;
15. At least one INR value (International Normalized Ratio); and
16. At least one pH value.

Accordingly, the sepsis score and/or the risk for sepsis may be determined based on one or more of, in particular all of, the aforementioned parameters or parameter values. Also a plurality of one or more of the aforementioned parameter values may be comprised in the set of patient and used to determine the at least one sepsis score.

In an example, the set of patient data is indicative of one or more of
1. At least one Patient age value;
2. At least one patient Heart rate value;
3. At least one Respiratory rate value;
4. At least one Diastolic blood pressure value;
5. At least one Systolic blood pressure value;
6. At least one Temperature value;
7. At least one Oxygen saturation value;
8. At least one WBC value;
9. At least one MDW value; and
   wherein the set of patient data is indicative of one or more of: a maximum lactate value of one or more lactate values, a last creatinine value of one or more creatinine values, a last lymphocyte value of one or more lymphocyte values, a mean lymphocyte value of a plurality of lymphocyte values, a minimum platelet value of one or more platelet values, a maximum neutrophil value of one or more neutrophil values, an initial INR value, a minimum pH value of one or more pH values, and a minimum creatinine value of one or more creatinine values. Accordingly, the sepsis score and/or the risk for sepsis may be determined based on one or more of, in particular all of, the aforementioned parameters or parameter values.

According to some embodiments, the computing device or ML model input data comprise or are based on a set of patient data. In particular, the set of patient data may be indicative of at least one of:
i. a plurality of patient Heart rate values;
ii. a plurality of Respiratory rate values;
iii. a plurality of Diastolic blood pressure values;
iv. a plurality of Systolic blood pressure values;
v. a plurality of Temperature values;
vi. a plurality of Oxygen saturation values;
vii. a plurality of WBC values; and
viii. a plurality of MDW values.

In particular, the set of patient data is indicative of a plurality of MDW values and at least one of the plurality of patient Heart rate values, the plurality of Respiratory rate values, the plurality of Diastolic blood pressure values, the plurality of Systolic blood pressure values, the plurality of Temperature values, the plurality of Oxygen saturation values and the plurality of WBC values.

According to some embodiments, the computing device or ML model input data comprise or are based on a set of patient data. In particular, the set of patient data may be indicative of at least one of:
i. a plurality of patient Heart rate values;
ii. a plurality of Respiratory rate values;
iii. a plurality of Diastolic blood pressure values;
iv. a plurality of Systolic blood pressure values;
v. a plurality of Temperature values;
vi. a plurality of Oxygen saturation values;
vii. a plurality of WBC values;
viii. a plurality of MDW values.
ix. a plurality of lactate values;
x. a plurality of creatinine values.
xi. a plurality of lymphocyte values,
xii. a plurality of platelet values;
xiii. a plurality of neutrophil values;
xiv. a plurality of e INR values; and
xv. a plurality of pH values.

According to the second aspect of the present disclosure, there is provided a computing device or system including one or more processors for data processing, wherein the computing device is configured to carry out steps of the method according to the first aspect of the present invention, as described hereinabove and hereinbelow.

The computing device may optionally comprise a data storage, for example storing at least a part of the set of patient data, data derived therefrom, the at least one sepsis score and/or information or data related thereto. Alternatively or additionally, software instructions or one or more computer programs may be stored at the data storage, which when executed by one or more processors of the computing device, may instruct the computing device to perform steps of the method described hereinabove and hereinbelow.

According to an embodiment, the computing device includes one or more machine learning models, for example implemented in a classifier circuitry or logic of the computing device, as described in more detail hereinabove and hereinbelow.

The computing device described herein may refer to any data processing device, including a standalone computing device or server and computing networks with a plurality of inter-operating computing devices, such as a cloud computing system or server system. Alternatively or additionally, the computing device may be embodied, at least in part, as mobile device, such as a smart phone a tablet computer, a notebook or the like.

The computing device may, in an example, comprise one or more communication interfaces configured to communicate with one or more remote devices, for example an external data storage or database, and/or one or more remote computing devices. For instance, at least a part of the set of patient data may be received via the communication interface from an external data storage. Alternatively or additionally, the computed at least one sepsis score or information related thereto may be transmitted to one or more remote devices or stored at the external data storage.

The third aspect of the present disclosure relates to a computer program, which, when executed by one or more processors of a computing device, instructs the computing device to perform steps of the method according to the first aspect of the present invention, as described hereinabove and hereinbelow.

The fourth aspect of the present disclosure relates to a computer-readable medium, for example a non-transitory computer-readable medium, storing the third aspect of the present disclosure.

These and other aspects of the disclosure will be apparent from and elucidated with reference to the appended figures, which may represent exemplary embodiments.

### BRIEF DESCRIPTION OF THE FIGURES

The subject-matter of the present disclosure will be explained in more detail in the following with reference to exemplary embodiments which are illustrated in the attached drawings, wherein:

FIG. 1 shows a computing device or system for determining, for a patient, a risk of developing sepsis according to an exemplary embodiment;

FIG. 2 shows a flow chart illustrating a method of determining, for a patient, a risk of developing sepsis according to an exemplary embodiment:

FIG. 3 illustrates steps of a method of determining, for a patient, a risk of developing sepsis according to an exemplary embodiment; and

FIG. 4 illustrates performance of a method of determining, for a patient, a risk of developing sepsis according to an exemplary embodiment.

FIG. 5 illustrates performance of a method of determining, for a patient, a risk of developing sepsis according to an exemplary embodiment.

FIG. 6 illustrates performance of a method of determining, for a patient, a risk of developing sepsis according to an exemplary embodiment.

The figures are schematic only and not true to scale. In principle, identical or like parts are provided with identical or like reference symbols in the figures.

### DETAILED DESCRIPTION OF THE INVENTION

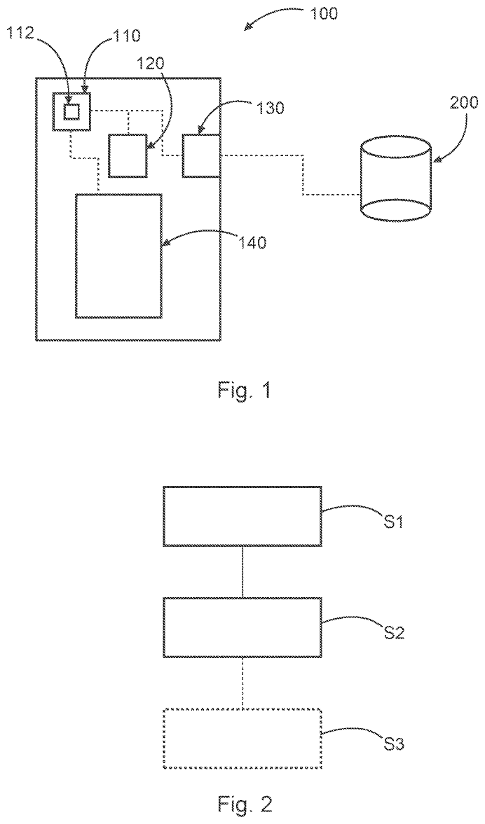

FIG. 1 shows a computing device or system 100, for example a clinical decision support system 100, configured to determine, for a patient, subject and/or individual, the risk for sepsis according to an exemplary embodiment. Alternatively or additionally, the computing device 100 may be configured to determine a patient-specific risk for sepsis and/or a patient-specific sepsis score. Alternatively or additionally, the computing device 100 may be configured to estimate and/or prognose the risk for sepsis, for example within a predetermined period of time.

The computing device 100 comprises a processing circuitry 110 or control circuitry 110 with one or more processors 112 for data processing. Optionally, the processing circuitry 110 or control circuitry 110 may include a classifier or a classifier circuitry. The computing device 100 further comprises at least one data storage 120 for storing data and/or software instructions. For example, at least a subset of the set of patient data and/or the at least one sepsis score may be stored at the data storage.

The exemplary computing device 100 of FIG. 1 further comprises at least one communication circuitry or interface 130 for communicatively coupling the computing device 100 to one or more external data sources 200 that may optionally store data and/or provide data to the computing device 100. The communication circuitry 130 may be configured for wired or wireless communication with the at least one external data source 200. It should be noted that the computing device 100 may comprise a plurality of communication circuits 130 or interfaces 130 for communicatively coupling the computing device 100 to a plurality of different external data sources 200.

The one or more external data sources 200 may for example be associated with one or more external servers communicatively coupled to the computing device 100, for example via the Internet, a LAN connection, a wireless connection or a wired connection. For example, the computing device 100 may be communicatively couplable to a hospital information system, a laboratory information system, a server of a health care provider, or any other server or data processing device.

As discussed in detail hereinabove and hereinbelow, the computing device 100 may be configured to determine the risk for sepsis to occur in a patient and/or may be configured to determine the patient-specific risk for developing (or already having) sepsis. Therein, the computing device 100 receives a set of patient data indicative of a patient age value, a patient heart rate value, a patient respiratory rate value, a patient diastolic blood pressure value, a patient systolic blood pressure value, a patient temperature value, a patient oxygen saturation value, a patient WBC value, and a MDW value of the patient. Further, the computing device 100 is configured to determine, based on processing the received set of patient data, at least one sepsis score indicative of the risk for a sepsis event or a sepsis onset occurring at the patient, for example within a predetermined period of time after the computation of the at least one sepsis score, such as within a predictive window of about 3 hours.

Optionally, the set of patient data is indicative of one or more of: a plurality of patient heart rate values, a plurality of patient respiratory rate values, a plurality of patient diastolic blood pressure values, a plurality of MDW values, a plurality of patient systolic blood pressure values, a plurality of patient temperature values, a plurality of patient oxygen saturation values, and a plurality of patient WBC values.

Therein, the plurality of values of a patient parameter may be obtained at the same or different time points. In particular, values of a plurality of values of a patient parameter, such as a plurality of heart rate values, can be obtained at different time points, thereby allowing to take into account the variation of the parameter over time and the effect or impact of said variation on the computed sepsis score.

Corresponding time information for each value of the plurality of values of a patient parameter may be included in the set of patient data, for example in the form of a timestamp associated with the respective value. The timestamps or time information of different values of said plurality of values may be identical, for example when two measurements of a parameter have been performed at the same time, or may differ, for example when two consecutive measurements of a parameter have been made.

Further optionally, the set of patient data is indicative of the plurality of patient heart rate values and determining the at least one sepsis score is based on a maximum value of the patient heart rate, a minimum value of the patient hearth rate, a most recent value of the patient heart rate and/or one or more mean values of the patient heart rate. Alternatively or additionally, the set of patient data can be indicative of the plurality of patient respiratory rate values and determining the at least one sepsis score is based on a maximum value of the patient respiratory rate, a minimum value of the patient respiratory rate, a most recent value of the patient respiratory rate and/or one or more mean values of the patient respiratory rate. Alternatively or additionally, the set of patient data can be indicative of the plurality of MDW values and determining the at least one sepsis score is based on a maximum value of MDW, a minimum value of MDW, a most recent value of MDW and/or one or more mean values of the MDW. Alternatively or additionally, the set of patient data can be indicative of the plurality of patient diastolic blood pressure values and determining the at least one sepsis score is based on a maximum value of the patient diastolic blood pressure, a minimum value of the patient diastolic blood pressure, a most recent value of the patient diastolic blood pressure and/or one or more mean values of the patient diastolic blood pressure. Alternatively or additionally, the set of patient data can be indicative of the plurality of patient systolic blood pressure values and determining the at least one sepsis score is based on a maximum value of the patient systolic blood pressure, a minimum value of the patient systolic blood pressure, a most recent value of the patient systolic blood pressure and/or one or more mean values of the patient systolic blood pressure. Alternatively or additionally, the set of patient data can be indicative of the plurality of patient oxygen saturation values and determining the at least one sepsis score is based on a maximum value of the patient oxygen saturation, a minimum value of the patient oxygen saturation, a most recent value of the patient oxygen saturation and/or one or more mean values of the patient oxygen saturation. Alternatively or additionally, the set of patient data can be indicative of the plurality of patient WBC values and determining the at least one sepsis score is based on a maximum value of the patient WBC, a minimum value of the patient WBC, a most recent value of the patient WBC, and/or one or more mean values of the patient WBC. Alternatively or additionally, the set of patient data is indicative of the plurality of patient temperature values and determining the at least one sepsis score can be based on a maximum value of the patient temperature, a minimum value of the patient temperature, a most recent value of the patient temperature and/or one or more mean values of the patient temperature.

Accordingly, any of the maximum parameter value(s), the minimum parameter value(s), the most recent value(s) and/or the mean value(s) can be computed by the computing device 100 to determine the sepsis score. Also, the sepsis score may be updated upon receiving one or more further parameter values of one or more parameters described herein as being usable for determining the sepsis score.

One or more of the aforementioned parameter values may be computed, determined and/or selected by the computing device 100 based on the obtained or received set of patient data. Accordingly, the method may comprise determining, computing and/or selecting a minimum value, a maximum value, a most recent value and/or a mean value among a plurality of values of a patient parameter, such as for example among the plurality of patient heart rate values, among the plurality of patient respiratory rate values, among the plurality of patient diastolic blood pressure values, among the plurality of MDW values, among the plurality of patient systolic blood pressure values, among the plurality of patient temperature values, among the plurality of patient oxygen saturation values, and/or among the plurality of patient WBC values. The same applies to any other parameter described herein as being potentially usable for computing the sepsis score. Alternatively or additionally, one or more of these values, i.e., the minimum value, the maximum value, the most recent value and/or the mean value of a plurality of values of a patient parameter, may be received as patient data at the computing device and used as input data to compute the sepsis score.

In the following, non-limiting examples of patient parameters that may be taken into consideration by the computing device 100 for determining the risk of sepsis are: age and pH value, and blood pressure. Accordingly, one or more of the age, at least one pH value and at least one blood pressure value may be taken into consideration.

The computing device 100 further includes a user interface 140 for receiving one or more user inputs. For instance, one or more parameter values or patient data may be provided to the computing device 100 via the user interface 140.

The user interface 140 may be configured to provide or output information to a user. For example, the computing device 100 may be configured to display the at least one sepsis score and/or generated information indicative of the likelihood or risk of sepsis and/or contextual information, such as one or more contributing parameters, at the user interface 140.

FIG. 2 shows a flow chart illustrating a method of determining a risk for developing sepsis according to an exemplary embodiment, for example using a computing device 100 as described with reference to FIG. 1.

Step S1 comprises receiving or obtaining, at the computing device 100, a set of patient data indicative of at least one of: a patient age value, a patient heart rate value, a patient respiratory rate value, a patient diastolic blood pressure value, a patient systolic blood pressure value, a patient temperature value, a patient oxygen saturation value, and a patient WBC value. Further, step S1 comprises receiving or obtaining, at the computing device 100, at least one value of a MDW of the patient.

Step S2 comprises determining, based on processing the received set of patient data with the computing device 100, at least one sepsis score indicative of the risk for a sepsis event occurring at the patient. Determining the sepsis risk or sepsis score may include computing and/or assessing the risk for sepsis. Assessing the sepsis risk may include determining a risk level or a tier for the patient, such as low, medium or high risk.

It is noted that one or more further optional steps, as described hereinabove in the summary part and with reference to FIG. 1, may be performed for determining the risk of sepsis in the patient and/or for determining one or more sepsis scores. For example, in an optional step S3 the at least one sepsis score and/or information indicative of a likelihood for the patient having or suffering from sepsis may optionally be displayed at the user interface 140 of the computing device 100. Alternatively or additionally, step S3 may comprise providing an output indicative of a tier for the patient at the user interface 140 of the computing device 100.

The determination of the sepsis risk or sepsis score may be carried out by the determination algorithm. The determination algorithm comprises the ML model and is configured to receive and process the set of patient data and to generate output data indicative of the sepsis risk or the sepsis score.

According to some embodiments, the input data of the determination algorithm comprise at least one of the following parameter values;
1. At least one Patient age (P) value;
2. At least one patient Heart rate (H) value;
3. At least one Respiratory rate (R) value;
4. At least one Diastolic blood pressure (D) value;
5. At least one Systolic blood pressure(S) value;
6. At least one Temperature (T) value;
7. At least one Oxygen saturation (O) value.

According to some embodiments, the input data of the determination algorithm comprise at least one of the following parameter values;
1. At least one Patient age (P) value;
2. At least one patient Heart rate (H) value;
3. At least one Respiratory rate (R) value;
4. At least one Diastolic blood pressure (D) value;
5. At least one Systolic blood pressure(S) value;
6. At least one Temperature (T) value;
7. At least one Oxygen saturation (O) value; and
8. At least one WBC value.

Furthermore, in particular, the input data of the determination algorithm can comprise at least one MDW value. In some embodiments, the set of patient data is indicative of all the values 1 To 7, or 1 to 8 listed above and the at least one MDW value may be the input data of the ML model, which processes said input data to generate output data indicative of the sepsis risk or the sepsis score.

According to some embodiments, the input data for the at least one ML model comprise or are based on a set of patient data. In particular, the set of patient data is indicative of one or more of:
1. At least one Patient age value;
2. At least one patient Heart rate value;
3. At least one Respiratory rate value;
4. At least one Diastolic blood pressure value;
5. At least one Systolic blood pressure value;
6. At least one Temperature value;
7. At least one Oxygen saturation value;
8. At least one WBC value;
9. At least one MDW value;
10. At least one lactate value;
11. At least one creatinine value,
12. At least one lymphocyte value,
13. At least one platelet value;
14. At least one neutrophil value;
15. At least one INR value (International Normalized Ratio); and
16. At least one pH value.

In some embodiments, the set of patient data is indicative of all the values 1 to 8, or 1 to 9 listed above and one or more of values 10 to 16.

In an example, the set of patient data is indicative of one or more of
1. At least one Patient age value;
2. At least one patient Heart rate value;
3. At least one Respiratory rate value;
4. At least one Diastolic blood pressure value;
5. At least one Systolic blood pressure value;
6. At least one Temperature value;
7. At least one Oxygen saturation value;
8. At least one WBC value;
9. At least one MDW value; and
   wherein the set of patient data is indicative of one or more of: a maximum lactate value of one or more lactate values, a last creatinine value of one or more creatinine values, a last lymphocyte value of one or more lymphocyte values, a mean lymphocyte value of a plurality of lymphocyte values, a minimum platelet value of one or more platelet values, a maximum neutrophil value of one or more neutrophil values, an initial INR value, a minimum pH value of one or more pH values, and a minimum creatinine value of one or more creatinine values. Accordingly, the sepsis score and/or the risk for sepsis may be determined based on one or more of, in particular all of, the aforementioned parameters or parameter values.

According to some embodiments, the input data of the determination algorithm comprise at least one of the following parameters values;
i. a plurality of patient Heart rate values: V_{H,1}, V_{H,2}, . . . , V_{H,n}_{_{H}};
ii. a plurality of Respiratory rate values: V_{R,1}, V_{R,2} . . . , V_{R,n}_{_{R}};
iii. a plurality of Diastolic blood pressure values: V_{D,1}, V_{D,2}, . . . , V_{D,}_{ₙ}_{D};
iv. a plurality of Systolic blood pressure values: V_{S,1}, V_{S,2}, . . . , V_{S,n}_{_{S}};
v. a plurality of Temperature values: V_{T,1}, V_{T,2}, . . . , V_{T,n}_{_{T}};
vi. a plurality of Oxygen saturation values: V_{O,1}, V_{O,2}, . . . , V_{O,n}_{_{O}};
vii. a plurality of WBC (W) values: V_{W,1}, V_{W,2}, . . . , V_{W,n}_{_{W}};
viii. a plurality of MDW (M) values: V_{M,1}, V_{M,2}, . . . , V_{M,n}_{_{M}},
   and the patient age value VA. For example, the values of each plurality of values i. to viii. listed above may be obtained at different time points and/or may have different timestamps. In other words, for each X∈χ={H, R, D, S, T, O, W, M} and for each j∈{1, 2, . . . , n_{X}}, the value V_{X,j} is obtained and/or recorded at a respective timepoint t_{X,j}. Moreover, for each X∈χ and for each i, j∈{1, 2, . . . , n_{X}} such that i≠j, the following relation holds: t_{X,i}≠t_{X,j}.

According to some embodiments, the ML model algorithm is configured to process 73 input values, i.e., in those embodiments, the input data of the ML model consist of 73 input parameters, i.e., the age value VA defined above and the following 72 input values;

$${I}_{X , 1} , {I}_{X , 2} , {I}_{X , 3} , {I}_{X , 4} , {I}_{X , 5} , {I}_{X , 6} , {I}_{X , 7} , {I}_{X , 8} , \\ {I}_{X , 9} , wherein X = H , R , D , S , T , O , W , M .$$

The determination algorithm or the ML model is configured to determine the 72 input values defined above by using the pluralities of values i. to viii. Described above as follows. In particular, for each for each X∈χ, the input parameters I_{X,1}, I_{X,2}, . . . , I_{X,9} are determined as follows:

$${I}_{X , 1} is the minimum value of {V}_{X , 1} , {V}_{X , 2} , ... , \\ {V}_{X , {n}_{X}} , i . e . , {I}_{X , 1} = min [ {V}_{X , 1} , {V}_{X , 2} , ... , {V}_{X , {n}_{X}} ] .$$

$${I}_{X , 2} is the maximum value of {V}_{X , 1} , {V}_{X , 2} , ... , \\ {V}_{X , {n}_{X}} , i . e . , {I}_{X , 2} = max [ {V}_{X , 1} , {V}_{X , 2} , ... , {V}_{X , {n}_{X}} ] .$$

I_{X,3} is the most recent value of V_{X,1}, V_{X,2}, . . . , V_{X,n}_{_{X}}. In particular, the I_{X,3} is the value of V_{X,1}, V_{X,2}, . . . , V_{X,n}_{_{X}} associated with the time point T₀≡max[t_{X,1}, t_{X,2}, . . . , t_{X,n}_{_{X}}].
I_{X,4} is an average value of V_{X,1}, V_{X,2}, . . . , V_{X,n}_{_{X}}, e.g.

$${I}_{X , 4} = \frac{{\sum }_{i = 1}^{{n}_{X}} {V}_{X , i}}{{n}_{X}} .$$

I_{X,5} is an average value of the values V_{X,1}, V_{X,2}, . . . , V_{X,n}_{_{X}} that have been obtained or recorded in the time window between T₁=T₀−60 minutes and T₀. In particular,

$${I}_{X , 5} = \frac{{\sum }_{i = 1}^{{n}_{X}} {ξ}_{X , i} {V}_{X , i}}{max [ {\sum }_{i = 1}^{{n}_{X}} {ξ}_{X , i} , 1 ]} ,$$

wherein ξ_{X,i} is equal to 1 if T₁≤ξ_{X,i}≤T₀ and is equal to 0 otherwise.
I_{X,6} is an average value of the values V_{X,1}, V_{X,2}, . . . , V_{X,n}_{_{X}} that have been obtained in the time window between T₂=T₁−60 minutes and T₁. In particular.

$${I}_{X , 6} = \frac{{\sum }_{i = 1}^{{n}_{X}} {δ}_{X , i} {V}_{X , i}}{max [ {\sum }_{i = 1}^{{n}_{X}} {δ}_{X , i} , 1 ]} ,$$

wherein δ_{X,i} is equal to 1 if T₂≤δ_{X,i}≤T₁ and is equal to 0) otherwise.
I_{X,7} is an average value of the values V_{X,1}, V_{X,2}, . . . , V_{X,n}_{_{X}} that have been obtained in the time window between T₃=T₂−60 minutes and T₂. In particular,

$${I}_{X , 7} = \frac{{\sum }_{i = 1}^{{n}_{X}} {θ}_{X , i} {V}_{X , i}}{max [ {\sum }_{i = 1}^{{n}_{X}} {θ}_{X , i} , 1 ]} ,$$

wherein δ_{X,i} is equal to 1 if T₃≤θ_{X,i}≤T₂ and is equal to 0 otherwise.

$${I}_{X , 8} = {I}_{X , 5} - {I}_{X , 6} .$$

$${I}_{X , 9} = {I}_{X , 6} - {I}_{X , 7} .$$

According to some embodiments, the ML model executed by the computing device 100 is configured to generate as output at least one sepsis score, the at least one sepsis score being a numerical score indicative of a patient's risk of sepsis within the predictive window, e.g. in the next 3 hours from a time of receipt of at least a subset of the set of patient data.

The ML model may be a supervised machine learning model trained to determine, for a patient, a risk of developing sepsis, i.e., at least one sepsis score indicative of the risk for a sepsis event occurring at the patient. In particular, the training of the ML model may be carried out by using a set of training data which may be labelled based on applying the following rules.

For instance, a positive sepsis label may be associated with or applied to an element of the set of training data, said element being associated with a respective training patient, when the clinical picture of the training patient meets all three of the following criteria, wherein sepsis onset time is the earliest time that the organ dysfunction criterion is met; provided Suspected Infection and SIRS criteria are also met within a 12-hour period:

Criterion 1: Suspected infection—Order of BOTH cultures and IV antibiotics must be met to be indicative of suspected infection, wherein the suspected infection time is the earliest time that either criteria is met, provided that (a) If antibiotics are ordered first, cultures must be obtained within 24 hours of first antibiotic dose delivered or (b) If cultures are ordered first, antibiotics are delivered within 72 hours of cultures being obtained (order for cultures (blood or otherwise), irrespective of result order for IV antibiotics with confirmed administration within 24 h prior to or within 72 h after culture order).

Criterion 2: Positive SIRS score ≥2 of criteria below met within 12 hours (before or after) of the suspected infection time, wherein SIRS time is the earliest time that at least 2 of the criteria below are met within a 12-hour window:
Temperature <36° C. (96.8° F.) or >38° C. (100.4° F.);
Heart rate >90 beats/min;
Respiratory rate >20 breaths/min or PaCO2<32 mm Hg;
WBC <4,000/μL or >12,000/μL, or >10% bands.

Criterion 3: Organ dysfunction—>1 of criteria below must be met within 12 hours (before or after) of the suspected infection time, wherein organ dysfunction time is the earliest time that at least one organ dysfunction criterion is met:
Hematologic:
   INR >1.5 or aPTT >60 seconds OR;
   Platelet count <100,000 cells/μL AND a ≥50% decline from baseline:
Metabolic: Lactate >2.0 mmol/L;
Cardiovascular:
   SBP <90 mmHg or MAP <65 mmHg with repeat confirmatory BP reading within
   15 min OR
   IV infusion of vasopressors (excluding OR push);
Liver: Total bilirubin ≥2 mg/dL AND doubling from baseline;
Kidney: Doubling of serum creatinine in patients WITHOUT history of CDK or ESRD:

### Respiratory:

PaO2/FiO2 ratio ≤300 OR SpO2/FiO2 ratio ≤315 OR;
Initiation of any mechanical ventilation;
Neurologic:
GCS <14 OR delirium OR
documented altered mental status (AMS) as reason for visit (excluding dementia or persistent vegetative state).

According to some embodiments, the ML model is configured to process the input data as described above. Exemplarily, the ML model comprises the XGBoost algorithm defined by Chen and Guestrin. A benefit of XGBoost algorithm is that it can deal with missing information without requiring data imputation.

In particular, according to an embodiment, the XGBoost algorithm has been trained by using at least 200,000 encounters of sepsis obtained from at least 2 large medical centers in the United States. For instance, the training data have been first cleaned and harmonized to ensure validity and consistency across all data sources e.g., uniform units used, data values within realistic ranges, extreme outliers, and other erroneous data removed, etc. The training dataset has been then labeled by using the sepsis criteria outlined above. Encounters in the training dataset which meet these sepsis criteria have been labeled as septic (the positive case), and those which do not meet these sepsis criteria have been labeled as negative (the negative case). The XGBoost model has been configured to process the input data described below. Standard nested K-fold cross-validation has been used to reduce bias, optimize the model, and improve generalizability, and the model is tested against the test dataset and locked when performance is deemed acceptable.

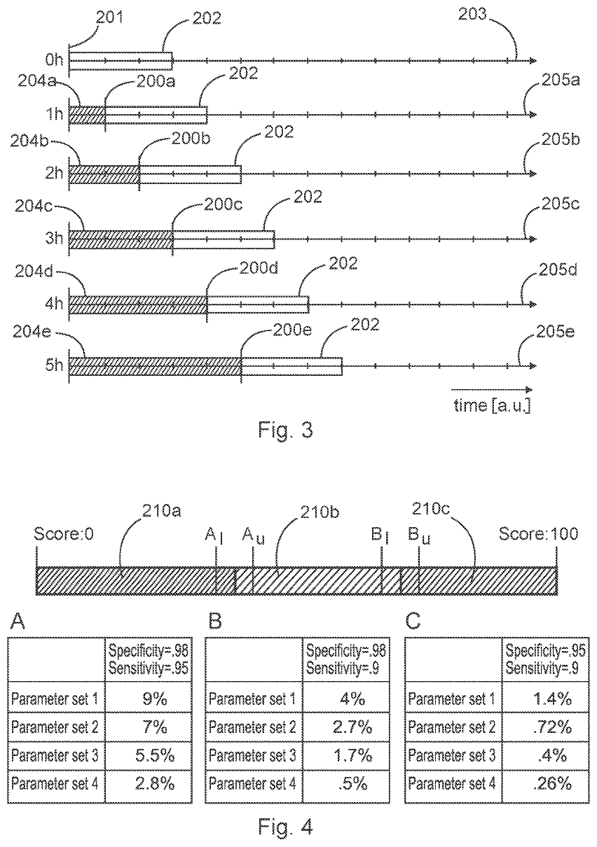

FIG. 3 illustrates steps of a method of determining, for a patient, a risk of developing sepsis according to an exemplary embodiment, for example using a computing device 100 as described with reference to FIG. 1.

In particular, FIG. 3 illustrates how a plurality of parameter values in the past, for example parameter values recorded, measured or obtained within a predetermined period of time 204a-204e prior to the time point 200a-200e at which an actual computation of the sepsis score is carried out, are taken into consideration in order to compute or predict the sepsis score for a predetermined period of time 204 after or following that actual computation of the sepsis score 200a-200e.

FIG. 3 illustrates in the uppermost row 203 the time of admission 201, for example at an emergency department, which may correspond to the time of computation of a first sepsis score. The computation of the first sepsis score is carried out by using first patient data comprising information indicative of a first patient age value, a first patient heart rate value, a first patient respiratory rate value, a first patient diastolic blood pressure value, a first patient systolic blood pressure value, a first patient temperature value, and a first patient oxygen saturation value. Optionally, one or more of a first WBC value and a first MDW value may be comprised in the first patient data. In particular, the first patient data may be processed by the determination algorithm which e.g. is configured to process the first patient data to generate the first sepsis score which is a numerical measure indicative of the risk for the sepsis event occurring at the patient within a predetermined period of time 202 from the time point at which the first sepsis score is computed 201.

Further. FIG. 3 shows in each of the rows 205a-205e a respective predetermined period of time 204a-204e prior to the respective timepoint 200a-200e at which a respective sepsis score at is determined. In particular, for each of the rows 205a-205e, the determination of the respective sepsis score is carried out by using the patient age and a respective plurality of values of patient heart rate, a respective plurality of values of patient respiratory rate, a respective plurality of values of patient diastolic blood pressure, a respective plurality of values of patient systolic blood pressure, a respective plurality of values of patient temperature, a respective plurality of values of patient oxygen saturation. In particular, said pluralities of values consist of values that are obtained within the respective predetermined period of time 204a-204e. These predetermined periods of time are also referred to as trending windows, and may for example have a maximum length of about 12 hours. Further, each of the rows 203, 205a-205e comprises the predetermined period of time 202 following the computation of the respective sepsis score. This predetermined period of time 202 is also referred to predictive window.

Optionally, in each of rows 205a-205e one or more WBC values and/or one or more MDW values may be comprised in the respective patient data and used to determine one or more further sepsis scores. Optionally one or more of at least one lactate value, at least one creatinine value, at least one lymphocyte value, at least one platelet value, at least one neutrophil value, at least one INR value, and at least one pH value may be comprised in the patient data and used to determine one or more sepsis scores, for example the first or one or more further sepsis scores.

In particular, for each row 205a-205e the ML model executed by the computing device 100 uses all patient data, i.e., parameter values associated with the patient, such as vitals, WBC and MDW, recorded in the past during the respective trending window 204a-204e to compute one or more of a maximum value, a minimum value, a most recent value, and/or a mean value of the parameter values of a particular parameter recorded within or obtained within, e.g. having a measurement time within the respective trending window 204a-204e. This of course may not apply to parameters that are constant over the time periods considered, such as the patient's age or gender. Further, one or more of these values may be used by the computing device 100 to compute a probability of sepsis onset within the predictive window 202, which may for example be around 3 hours.

For example, the ML model executed by the computing device 100 can be configured to consider patient data over a period of up to 12 hours in the past in the trending window 204 and to predict sepsis onset 3 hours in advance for the predictive window 202.

Accordingly, in each of the rows 203, 205a-205e shown in FIG. 3, at least a first sepsis score may be computed. In particular, in the uppermost row 203, the first patient data may be used to compute said first sepsis score. Subsequently, more parameter values may be received at the computing device and used to compute a further sepsis score in each of the row 205a-205e shown in FIG. 3 and/or may be used to update or re-compute the at least first sepsis score.

Optionally, the ML model executed by the computing device 100 may stop computing the sepsis risk score where the patient is discharged from the hospital, or when the patient is transferred to the ICU, operating room or post-op recovery ward.

FIG. 4 illustrates steps of a method of determining, for a patient, a risk of developing sepsis according to an exemplary embodiment, for example using a computing device 100 as described with reference to FIG. 1.

FIG. 4 illustrates determining, based on the at least one sepsis score, a sepsis risk level 210a. 210b. 210c. In particular the sepsis risk score can be a numerical measure between 0 and 100, and can exemplarily be stratified into three risk tiers-low 210a, medium 210b, and high 210c, as depicted in FIG. 4. More specifically. FIG. 4 shows or illustrates shrinkage of the medium risk zone 210b or intermediate zone 210b as a function of model complexity and/or a function of the parameters or features considered to compute the sepsis score. Therein, each of the tables A, B and C illustrates performance gains achieved at three sets of specificity and sensitivity targets for four ML models trained on four different sets of features or parameters, which are referred to as parameter sets 1 to 4. Systematically adding the features to model training consistently reduced the size of the indeterminate zone 210b, expressed as a percentage of all encounters considered tables A, B, C of FIG. 4. Tables A, B, and C of FIG. 4 are reproduced here.

Table A of FIG. 4.

| | Specificity = .98 |
|---|---|
| | Sensitivity = .95 |
| Parameter set 1 | 9% |
| Parameter set 2 | 7% |
| Parameter set 3 | 5.5% |
| Parameter set 4 | 2.8% |

Table B of FIG. 4.

| | Specificity = .98 |
|---|---|
| | Sensitivity = .95 |
| Parameter set 1 | 4% |
| Parameter set 2 | 2.7% |
| Parameter set 3 | 1.7% |
| Parameter set 4 | .5% |

Table C of FIG. 4.

| | Specificity = .98 |
|---|---|
| | Sensitivity = .95 |
| Parameter set 1 | 1.4% |
| Parameter set 2 | .72% |
| Parameter set 3 | .4% |
| Parameter set 4 | .26% |

The specificity and sensitivity targets shown in FIG. 4 include a first lower sensitivity target A_1=0.95, an upper sensitivity target A_u=0.90, a lower specificity target B_I=0.95, and an upper specificity target B_u=0.98. The three sets of specificity and sensitivity targets for panels A, B and C shown in FIG. 4 are as follows: Panel A shows the indeterminate zone 210b shrinkage as a function of model complexity for A_I and B_u threshold positions or targets in sensitivity and specificity. Panel B shows the same for A_u and B_u thresholds or targets in sensitivity and specificity. Panel C shows the same for for A_u and B_I thresholds targets in sensitivity and specificity. As one moves from the left column A across column B to column C in the tables of FIG. 4, there is an overall shrinkage of indeterminate zone size for all models shown. This is a consequence of relaxing the specificity and sensitivity requirements defined.

The four parameter sets on which the ML models were trained are as follows: Parameter set 1 includes at least one Patient age value, at least one patient Heart rate value, at least one Respiratory rate value, at least one Diastolic blood pressure value, at least one Systolic blood pressure value, at least one Temperature value, and at least one Oxygen saturation value. Parameter set 2 includes parameter set 1 and at least one WBC value. Parameter set 3 includes parameter set 2 and at least one MDW value. Parameter set 4 includes parameter set 3 and at least one lactate value, at least one creatinine value, at least one lymphocyte value, at least one platelet value, at least one neutrophil value, at least one INR value, and at least one pH value.

As mentioned, from parameter sets 1 to 4, complexity of the ML models is increased and consistently the size of the indeterminate zone 210b, expressed as a percentage of all encounters considered tables A, B. C of FIG. 4, is reduced for each of the three sets of specificity and sensitivity targets. Hence, by applying the method described herein, the number of patients who are erroneously diagnosed as sepsis patients can be reduced, due to the shrinkage of the intermediate zone 210b.

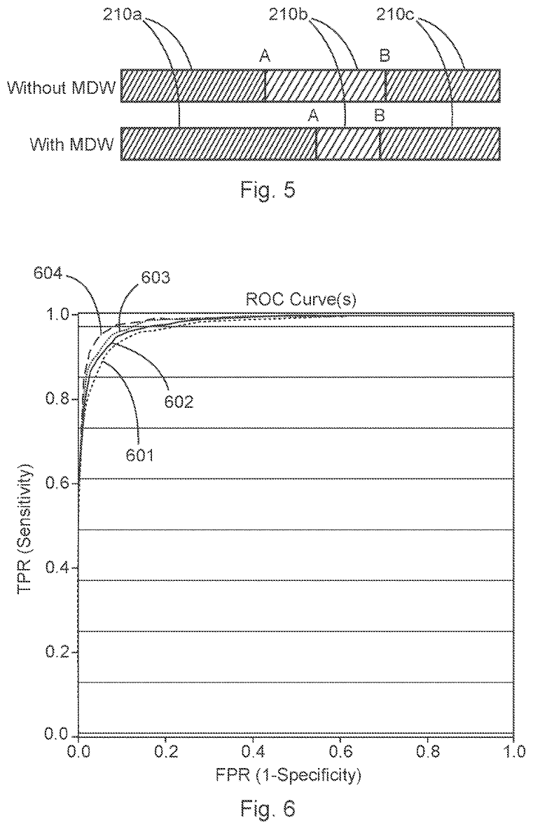

FIG. 5 illustrates steps of a method of determining, for a patient, a risk of developing sepsis according to an exemplary embodiment, for example using a computing device 100 as described with reference to FIG. 1. In particular, FIG. 5 shows a comparison of determining the risk for sepsis without MDW (only Vitals and WBC values fed to model) and with MDW (model provided with Vitals. WBC and MDW values). As can be seen, when MDW is taken into consideration, the medium risk zone 210b is reduced in size, such that patients in the medium risk region or zone 210b can be reduced by about 40%. This shows that risk stratification, e.g. the ability to clearly differentiate between low sepsis risk patients 210a and high sepsis risk patients 210c, is significantly improved when applying the method described herein.

FIG. 5 specifically illustrates the distribution of patients in risk tiers using a 3-tier ML model with a low-risk cutoff A optimized for high Specificity >98% and a high-risk cutoff B optimized for high Sensitivity >95% showing a significantly reduced medium risk group and improved classification to the low and high-risk groups when MDW is available. In both cases, statistics were computed for the same cohort of patients with orders for Complete Blood Count with Differential (CBC-diff), an assay for counts of red blood cells, white blood cells and platelets together with type differentiation of white blood cells (monocytes, neutrophils, lymphocytes, basophils and eosinophils). Data obtained from this test may be used to calculate MDW.

FIG. 6 illustrates performance of a method of determining, for a patient, a risk of developing sepsis according to an exemplary embodiment. Specifically, FIG. 6 shows receiver-operating-characteristic. ROC, curves 601-604 illustrating performance of a method of determining, for a patient, a risk of developing sepsis according to an exemplary embodiment.

Generally, an ROC curve is a graph showing the performance of a classification model at all classification thresholds, wherein the True Positive Rate. TPR, is plotted versus the False Positive Rate, FPR. The TPR is defined as the number of True Positives (TP), i.e., an outcome where the ML model correctly predicts the positive class, divided by the sum of True Positives and False Negatives (FN). i.e., an outcome where the ML model incorrectly predicts the negative class: TPR=TP/(TP+FN). The FPR, on the other hand is defined as the number of False Positives (FP), i.e., an outcome where the ML model incorrectly predicts the positive class, divided by the sum of False Positives and True Negatives (TN), i.e., an outcome where the ML model correctly predicts the negative class: FPR=FP/(FP+TN). Likewise, TPR can be referred to as sensitivity and FPR can be referred to as (1-specificity).

The ROC curves 601-604 correspond to ML models considering a different number of input parameters or parameter values. Specifically, the four ROC curves 601-604 of FIG. 6 correspond to the four ML models trained on four different sets of features or parameters, namely parameter sets 1 to 4 of FIG. 4. In particular. ROC curve 601 corresponds to parameter set 1, ROC curve 602 corresponds to parameter set 2, ROC curve 603 corresponds to parameter set 3, and ROC curve 604 corresponds to parameter set 4 as defined in FIG. 4.

The four parameter sets on which the ML models were trained are as follows: Parameter set 1 includes at least one Patient age value, at least one patient Heart rate value, at least one Respiratory rate value, at least one Diastolic blood pressure value, at least one Systolic blood pressure value, at least one Temperature value, and at least one Oxygen saturation value. Parameter set 2 includes parameter set 1 and at least one WBC value. Parameter set 3 includes parameter set 2 and at least one MDW value. Parameter set 4 includes parameter set 3 and at least one lactate value, at least one creatinine value, at least one lymphocyte value, at least one platelet value, at least one neutrophil value, at least one INR value, and at least one pH value.

The two-dimensional area under the curve. AUC, under the ROC curve of FIG. 6 provides an aggregate measure of the performance of the respective ML model. The ROC-AUC value for ROC curve 1 and parameter set 1 is 0.975, the ROC-AUC value for ROC curve 2 and parameter set 2 is 0.981, the ROC-AUC value for ROC curve 3 and parameter set 3 is 0.984, and the ROC-AUC value for ROC curve 4 and parameter set 4 is 0.988. Hence, adding parameters or parameter values from parameter sets 1 through 4 increases performance of the respective ML model.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### EXEMPLARY ASPECTS

The following exemplary Aspects are provided, the numbering of which is not to be construed as designating levels of importance:

Aspect 1 provides a computer-implemented method of determining, for a patient, a risk of developing sepsis, the method comprising:
obtaining, at a computing device, a set of patient data indicative of:
   at least one of: a patient age value, a patient heart rate value, a patient respiratory rate value, a patient diastolic blood pressure value, a patient systolic blood pressure value, a patient temperature value, and a patient oxygen saturation value; and
   at least one value related to a Monocyte Distribution Width (MDW) of the patient; and
   determining with the computing device, based on the obtained set of patient data, at least one sepsis score indicative of the risk for a sepsis event occurring at the patient.

Aspect 2 provides the method according to Aspect 1, wherein the set of patient data is indicative of at least one of: a patient age value, a patient heart rate value, a patient respiratory rate value, a patient diastolic blood pressure value, a patient systolic blood pressure value, a patient temperature value, and a patient oxygen saturation value, and a patient White Blood cell Count (WBC) value.

Aspect 3 provides the method according to any one of Aspects 1-2, wherein determining the at least one sepsis score includes computing the risk for the sepsis event occurring at the patient based on at least one machine learning model, wherein the machine learning model is configured to receive and process input data, the input data comprising and/or being based on the set of patient data.

Aspect 4 provides the method according to Aspect 3, wherein the at least one machine learning model comprises a trained gradient boosting algorithm, a trained artificial neural network, a trained feed forward neural network, a trained convolutional neural network and/or a trained deep neural network.

Aspect 5 provides the method according to any one of Aspects 1-4, wherein the set of patient data is indicative of at least one of: a plurality of patient heart rate values, a plurality of patient respiratory rate values, a plurality of patient diastolic blood pressure values, a plurality of patient systolic blood pressure values, a plurality of patient temperature values, a plurality of patient oxygen saturation values, and a plurality of patient WBC values.

Aspect 6 provides the method according to Aspect 5, wherein:
the set of patient data is indicative of the plurality of patient heart rate values and determining the at least one sepsis score is based on a maximum value of the patient heart rate, a minimum value of the patient hearth rate, a most recent value of the patient heart rate and/or one or more mean values of the patient heart rate; or
the set of patient data is indicative of the plurality of patient respiratory rate values and determining the at least one sepsis score is based on a maximum value of the patient respiratory rate, a minimum value of the patient respiratory rate, a most recent value of the patient respiratory rate and/or one or more mean values of the patient respiratory rate; or
the set of patient data is indicative of the plurality of patient diastolic blood pressure values and determining the at least one sepsis score is based on a maximum value of the patient diastolic blood pressure, a minimum value of the patient diastolic blood pressure, a most recent value of the patient diastolic blood pressure and/or one or more mean values of the patient diastolic blood pressure; or
the set of patient data is indicative of the plurality of patient systolic blood pressure values and determining the at least one sepsis score is based on a maximum value of the patient systolic blood pressure, a minimum value of the patient systolic blood pressure, a most recent value of the patient systolic blood pressure and/or one or more mean values of the patient systolic blood pressure; or
the set of patient data is indicative of the plurality of patient oxygen saturation values and determining the at least one sepsis score is based on a maximum value of the patient oxygen saturation, a minimum value of the patient oxygen saturation a most recent value of the patient oxygen saturation and/or one or more mean values of the patient oxygen saturation; or
the set of patient data is indicative of the plurality of patient WBC values and determining the at least one sepsis score is based on a maximum value of the patient WBC, a minimum value of the patient WBC a most recent value of the patient WBC, and/or one or more mean values of the patient WBC; or
the set of patient data is indicative of the plurality of patient temperature values and determining the at least one sepsis score is based on a maximum value of the patient temperature, a minimum value of the patient temperature, a most recent value of the patient temperature and/or one or more mean values of the patient temperature; or
a combination thereof.

Aspect 7 provides the method according to Aspect 6 when combined with Aspect 3 or 4, wherein:
the input data comprise the maximum value of the patient heart rate the minimum value of the patient hearth rate, the most recent value of the patient hearth rate and/or the one or more mean values of the patient heart rate; or
the input data comprise the maximum value of the patient respiratory rate, the minimum value of the patient respiratory rate, the most recent value of the patient respiratory rate and/or the one or more mean values of the patient respiratory rate; or
the input data comprise the maximum value of the patient diastolic blood pressure, the minimum value of the patient diastolic blood pressure, the most recent value of the patient diastolic blood pressure and/or the one or more mean values of the patient diastolic blood pressure; or
the input data comprise the maximum value of the patient systolic blood pressure, the minimum value of the patient systolic blood pressure the most recent value of the patient systolic blood pressure and/or the one or more mean values of the patient systolic blood pressure; or
the input data comprise the maximum value of the patient oxygen saturation, the minimum value of the patient oxygen saturation, the most recent value of the patient oxygen saturation and/or the one or more mean values of the patient oxygen saturation; or
the input data comprise the maximum value of the patient WBC, the minimum value of the patient WBC, the most recent value of the patient WBC and/or the one or more mean values of the patient WBC; or
the input data comprise the maximum value of the patient temperature, the minimum value of the patient temperature, the most recent value of the patient temperature and/or the one or more mean values of the patient temperature; or
a combination thereof.

Aspect 8 provides the method according to any one of Aspects 1-7, wherein the set of patient data is indicative of a plurality of MDW values of the patient and determining the at least one sepsis score is based on a maximum value of the MDW of the patient, a minimum value of the MDW of the patient, a most recent value of the MDW of the patient and/or one or more mean values of the MDW of the patient.

Aspect 9 provides the method according to any one of Aspects 1-8, wherein the at least one sepsis score is a numerical measure indicative of the risk for the sepsis event occurring at the patient within a predetermined period of time.

Aspect 10 provides the method according to Aspect 9, wherein the at least one sepsis score is a numerical measure indicative of the risk for a sepsis event occurring at the patient within three hours from a time of receipt of at least a subset of the set of patient data.

Aspect 11 provides the method according to any one of Aspects 1-10, further comprising determining, based on the at least one sepsis score, a sepsis risk level.

Aspect 12 provides the method according to Aspect 11, wherein determining the sepsis risk level comprises selecting the sepsis risk level from a plurality of risk levels.

Aspect 13 provides the method according to Aspect 12, wherein selecting the sepsis risk level from the plurality of risk levels comprises comparing the at least one sepsis score with one or more risk score thresholds.

Aspect 14 provides the method according to Aspect 13, wherein the plurality of risk level comprises a first risk level, a second risk level and a third risk level, wherein:
if the at least one sepsis score is lower than a first threshold, the first risk level is selected as sepsis risk level,
if the at least one sepsis score is greater than the first threshold and lower than a second threshold, the second risk level is selected as sepsis risk level, and
if the at least one sepsis score is greater than the second threshold, the third risk level is selected as sepsis risk level.

Aspect 15 provides the method according to any one of Aspects 1-14, wherein the set of patient data comprises the patient age value, the patient heart rate value, the patient respiratory rate value, the patient diastolic blood pressure value, the patient systolic blood pressure value, the patient temperature value, the patient oxygen saturation value and the patient WBC value.

Aspect 16 provides the method according to any one of Aspects 1-15, wherein the set of patient data comprises the patient heart rate value, the patient respiratory rate value, the patient diastolic blood pressure value, the patient systolic blood pressure value, the patient temperature value and the patient oxygen saturation value.

Aspect 17 provides a computing device including one or more processors for data processing, wherein the computing device is configured to carry out steps of the method according to any one of Aspects 1-16.

Aspect 18 provides a computer program, which, when executed by one or more processors of a computing device, instructs the computing device to perform steps of the method according to any one of Aspects 1 to 16.

Aspect 19 provides a non-transitory computer-readable medium storing a computer program according to Aspect 18.

Aspect 20 provides a computer-implemented method of determining, for a patient, a risk of developing sepsis, the method comprising:
obtaining, at a computing device, a set of patient data indicative of:
   at least one of: a patient age value, a patient heart rate value, a patient respiratory rate value, a patient diastolic blood pressure value, a patient systolic blood pressure value, a patient temperature value, a patient oxygen saturation value, and a patient white blood count (WBC) value; and
   at least one value of a Monocyte Distribution Width (MDW) of the patient; and
   determining, based on processing the obtained set of patient data with the computing device, at least one sepsis score indicative of the risk for a sepsis event occurring at the patient.

Aspect 21 provides the method according to Aspect 20, wherein determining the at least one sepsis score includes computing the risk for the sepsis event occurring at the patient based on at least one machine learning model of the computing device, wherein the machine learning model is configured to receive and process input data, the input data comprising and/or being based on the set of patient data.

Aspect 22 provides the method according to Aspect 21, wherein the at least one machine learning model comprises a trained gradient boosting algorithm, a trained artificial neural network, a trained feed forward neural network, a trained convolutional neural network and/or a trained deep neural network.

Aspect 23 provides the method according to any one of Aspects 20-22, wherein the set of patient data is indicative of at least one of: a plurality of patient heart rate values, a plurality of patient respiratory rate values, a plurality of patient diastolic blood pressure values, a plurality of patient systolic blood pressure values, a plurality of patient temperature values, a plurality of patient oxygen saturation values, and a plurality of patient WBC values.

Aspect 24 provides the method according to Aspect 23, wherein:
the set of patient data is indicative of the plurality of patient heart rate values and determining the at least one sepsis score is based on a maximum value of the patient heart rate, a minimum value of the patient hearth rate, a most recent value of the patient heart rate and/or one or more mean values of the patient heart rate; or
the set of patient data is indicative of the plurality of patient respiratory rate values and determining the at least one sepsis score is based on a maximum value of the patient respiratory rate, a minimum value of the patient respiratory rate, a most recent value of the patient respiratory rate and/or one or more mean values of the patient respiratory rate; or
the set of patient data is indicative of the plurality of patient diastolic blood pressure values and determining the at least one sepsis score is based on a maximum value of the patient diastolic blood pressure, a minimum value of the patient diastolic blood pressure, a most recent value of the patient diastolic blood pressure and/or one or more mean values of the patient diastolic blood pressure; or
the set of patient data is indicative of the plurality of patient systolic blood pressure values and determining the at least one sepsis score is based on a maximum value of the patient systolic blood pressure, a minimum value of the patient systolic blood pressure, a most recent value of the patient systolic blood pressure and/or one or more mean values of the patient systolic blood pressure; or
the set of patient data is indicative of the plurality of patient oxygen saturation values and determining the at least one sepsis score is based on a maximum value of the patient oxygen saturation, a minimum value of the patient oxygen saturation a most recent value of the patient oxygen saturation and/or one or more mean values of the patient oxygen saturation; or
the set of patient data is indicative of the plurality of patient WBC values and determining the at least one sepsis score is based on a maximum value of the patient WBC, a minimum value of the patient WBC a most recent value of the patient WBC, and/or one or more mean values of the patient WBC; or
the set of patient data is indicative of the plurality of patient temperature values and determining the at least one sepsis score is based on a maximum value of the patient temperature, a minimum value of the patient temperature, a most recent value of the patient temperature and/or one or more mean values of the patient temperature; or
a combination thereof.

Aspect 25 provides the method according to Aspect 24 when combined with Aspect 21 or 22, wherein:
the input data comprise the maximum value of the patient heart rate the minimum value of the patient hearth rate, the most recent value of the patient hearth rate and/or the one or more mean values of the patient heart rate; or
the input data comprise the maximum value of the patient respiratory rate, the minimum value of the patient respiratory rate, the most recent value of the patient respiratory rate and/or the one or more mean values of the patient respiratory rate; or
the input data comprise the maximum value of the patient diastolic blood pressure, the minimum value of the patient diastolic blood pressure, the most recent value of the patient diastolic blood pressure and/or the one or more mean values of the patient diastolic blood pressure; or
the input data comprise the maximum value of the patient systolic blood pressure, the minimum value of the patient systolic blood pressure the most recent value of the patient systolic blood pressure and/or the one or more mean values of the patient systolic blood pressure; or
the input data comprise the maximum value of the patient oxygen saturation, the minimum value of the patient oxygen saturation, the most recent value of the patient oxygen saturation and/or the one or more mean values of the patient oxygen saturation; or
the input data comprise the maximum value of the patient WBC, the minimum value of the patient WBC, the most recent value of the patient WBC and/or the one or more mean values of the patient WBC; or
the input data comprise the maximum value of the patient temperature, the minimum value of the patient temperature, the most recent value of the patient temperature and/or the
one or more mean values of the patient temperature; or a combination thereof.

Aspect 26 provides the method according to any one of Aspects 20-25, wherein the set of patient data is indicative of a plurality of MDW values of the patient and determining the at least one sepsis score is based on a maximum value of the MDW of the patient, a minimum value of the MDW of the patient, a most recent value of the MDW of the patient and/or one or more mean values of the MDW of the patient.

Aspect 27 provides the method according to any one of Aspects 20-26, wherein the at least one sepsis score is a numerical measure indicative of the risk for the sepsis event occurring at the patient within a predetermined period of time.

Aspect 28 provides the method according to Aspect 27, wherein the at least one sepsis score is a numerical measure indicative of the risk for a sepsis event occurring at the patient within three hours from a time of receipt of at least a subset of the set of patient data.

Aspect 29 provides the method according to any one of Aspects 20-28, further comprising determining, based on the at least one sepsis score, a sepsis risk level.

Aspect 30 provides the method according to Aspect 29, wherein determining the sepsis risk level comprises selecting the sepsis risk level from a plurality of risk levels.

Aspect 31 provides the method according to Aspect 30, wherein selecting the sepsis risk level from the plurality of risk levels comprises comparing the at least one sepsis score with one or more risk score thresholds.

Aspect 32 provides the method according to Aspect 31, wherein the plurality of risk level comprises a first risk level, a second risk level and a third risk level, wherein:
if the at least one sepsis score is lower than a first threshold, the first risk level is selected as sepsis risk level,
if the at least one sepsis score is greater than the first threshold and lower than a second threshold, the second risk level is selected as sepsis risk level, and
if the at least one sepsis score is greater than the second threshold, the third risk level is selected as sepsis risk level.

Aspect 33 provides the method according to any one of Aspects 20-32, wherein the set of patient data comprises the patient age value, the patient heart rate value, the patient respiratory rate value, the patient diastolic blood pressure value, the patient systolic blood pressure value, the patient temperature value, the patient oxygen saturation value and the patient white blood count value.

Aspect 34 provides the method according to any one of Aspects 20-33, wherein the set of patient data comprises the patient heart rate value, the patient respiratory rate value, the patient diastolic blood pressure value, the patient systolic blood pressure value, the patient temperature value and the patient oxygen saturation value.

Aspect 35 provides a computing device including one or more processors for data processing, wherein the computing device is configured to carry out steps of the method according to any one of Aspects 20-34.

Aspect 36 provides a computer program, which, when executed by one or more processors of a computing device, instructs the computing device to perform steps of the method according to any one of Aspects 20 to 34.

Aspect 37 provides a non-transitory computer-readable medium storing a computer program according to Aspect 36.

Aspect 38 provides the method, computing device, computer program, or non-transitory computer-readable medium of any one or any combination of Aspects 1-37 optionally configured such that all elements or options recited are available to use or select from.

1. A computer-implemented method of determining, for a patient, a risk of developing sepsis, the method comprising:
obtaining, at a computing device, a set of patient data indicative of:
at least one of: a patient age value, a patient heart rate value, a patient respiratory rate value, a patient diastolic blood pressure value, a patient systolic blood pressure value, a patient temperature value, and a patient oxygen saturation value; and
at least one value related to a Monocyte Distribution Width (MDW) of the patient; and
determining with the computing device, based on the obtained set of patient data, at least one sepsis score indicative of the risk for a sepsis event occurring at the patient.

2. The method according to claim 1, wherein the set of patient data is indicative of at least one of: a patient age value, a patient heart rate value, a patient respiratory rate value, a patient diastolic blood pressure value, a patient systolic blood pressure value, a patient temperature value, and a patient oxygen saturation value, and a patient White Blood cell Count (WBC) value.

3. The method according to claim 1, wherein determining the at least one sepsis score includes computing the risk for the sepsis event occurring at the patient based on at least one machine learning model, wherein the machine learning model is configured to receive and process input data, the input data comprising and/or being based on the set of patient data.

4. The method according to claim 3, wherein the at least one machine learning model comprises a trained gradient boosting algorithm, a trained artificial neural network, a trained feed forward neural network, a trained convolutional neural network and/or a trained deep neural network.

5. The method according to claim 1, wherein the set of patient data is indicative of at least one of: a plurality of patient heart rate values, a plurality of patient respiratory rate values, a plurality of patient diastolic blood pressure values, a plurality of patient systolic blood pressure values, a plurality of patient temperature values, a plurality of patient oxygen saturation values, and a plurality of patient WBC values.

6. The method according to claim 5, wherein:
the set of patient data is indicative of the plurality of patient heart rate values and determining the at least one sepsis score is based on a maximum value of the patient heart rate, a minimum value of the patient hearth rate, a most recent value of the patient heart rate and/or one or more mean values of the patient heart rate; or
the set of patient data is indicative of the plurality of patient respiratory rate values and determining the at least one sepsis score is based on a maximum value of the patient respiratory rate, a minimum value of the patient respiratory rate, a most recent value of the patient respiratory rate and/or one or more mean values of the patient respiratory rate; or the set of patient data is indicative of the plurality of patient diastolic blood pressure values and determining the at least one sepsis score is based on a maximum value of the patient diastolic blood pressure, a minimum value of the patient diastolic blood pressure, a most recent value of the patient diastolic blood pressure and/or one or more mean values of the patient diastolic blood pressure; or
the set of patient data is indicative of the plurality of patient systolic blood pressure values and determining the at least one sepsis score is based on a maximum value of the patient systolic blood pressure, a minimum value of the patient systolic blood pressure, a most recent value of the patient systolic blood pressure and/or one or more mean values of the patient systolic blood pressure; or
the set of patient data is indicative of the plurality of patient oxygen saturation values and determining the at least one sepsis score is based on a maximum value of the patient oxygen saturation, a minimum value of the patient oxygen saturation a most recent value of the patient oxygen saturation and/or one or more mean values of the patient oxygen saturation; or
the set of patient data is indicative of the plurality of patient WBC values and determining the at least one sepsis score is based on a maximum value of the patient WBC, a minimum value of the patient WBC a most recent value of the patient WBC, and/or one or more mean values of the patient WBC; or
the set of patient data is indicative of the plurality of patient temperature values and determining the at least one sepsis score is based on a maximum value of the patient temperature, a minimum value of the patient temperature, a most recent value of the patient temperature and/or one or more mean values of the patient temperature; or
a combination thereof.

7. The method according to claim 6, wherein:
the input data comprise the maximum value of the patient heart rate the minimum value of the patient hearth rate, the most recent value of the patient hearth rate and/or the one or more mean values of the patient heart rate; or
the input data comprise the maximum value of the patient respiratory rate, the minimum value of the patient respiratory rate, the most recent value of the patient respiratory rate and/or the one or more mean values of the patient respiratory rate; or
the input data comprise the maximum value of the patient diastolic blood pressure, the minimum value of the patient diastolic blood pressure, the most recent value of the patient diastolic blood pressure and/or the one or more mean values of the patient diastolic blood pressure; or
the input data comprise the maximum value of the patient systolic blood pressure, the minimum value of the patient systolic blood pressure the most recent value of the patient systolic blood pressure and/or the one or more mean values of the patient systolic blood pressure; or
the input data comprise the maximum value of the patient oxygen saturation, the minimum value of the patient oxygen saturation, the most recent value of the patient oxygen saturation and/or the one or more mean values of the patient oxygen saturation; or
the input data comprise the maximum value of the patient WBC, the minimum value of the patient WBC, the most recent value of the patient WBC and/or the one or more mean values of the patient WBC; or
the input data comprise the maximum value of the patient temperature, the minimum value of the patient temperature, the most recent value of the patient temperature and/or the one or more mean values of the patient temperature; or
a combination thereof.

8. The method according to claim 1, wherein the set of patient data is indicative of a plurality of MDW values of the patient and determining the at least one sepsis score is based on a maximum value of the MDW of the patient, a minimum value of the MDW of the patient, a most recent value of the MDW of the patient and/or one or more mean values of the MDW of the patient.

9. The method according to claim 1, wherein the at least one sepsis score is a numerical measure indicative of the risk for the sepsis event occurring at the patient within a predetermined period of time.

10. The method according to claim 9, wherein the at least one sepsis score is a numerical measure indicative of the risk for a sepsis event occurring at the patient within three hours from a time of receipt of at least a subset of the set of patient data.

11. The method according to claim 1, further comprising determining, based on the at least one sepsis score, a sepsis risk level.

12. The method according to claim 11, wherein determining the sepsis risk level comprises selecting the sepsis risk level from a plurality of risk levels.

13. The method according to claim 12, wherein selecting the sepsis risk level from the plurality of risk levels comprises comparing the at least one sepsis score with one or more risk score thresholds.

14. The method according to claim 13, wherein the plurality of risk level comprises a first risk level, a second risk level and a third risk level, wherein:
if the at least one sepsis score is lower than a first threshold, the first risk level is selected as sepsis risk level,
if the at least one sepsis score is greater than the first threshold and lower than a second threshold, the second risk level is selected as sepsis risk level, and
if the at least one sepsis score is greater than the second threshold, the third risk level is selected as sepsis risk level.

15. The method according to claim 1, wherein the set of patient data comprises the patient age value, the patient heart rate value, the patient respiratory rate value, the patient diastolic blood pressure value, the patient systolic blood pressure value, the patient temperature value, the patient oxygen saturation value and the patient WBC value.

16. The method according to claim 1, wherein the set of patient data comprises the patient heart rate value, the patient respiratory rate value, the patient diastolic blood pressure value, the patient systolic blood pressure value, the patient temperature value and the patient oxygen saturation value.

17. A computing device including one or more processors for data processing, wherein the computing device is configured to carry out steps of the method according to claim 1.

18. A computer program, which, when executed by one or more processors of a computing device, instructs the computing device to perform steps of the method according to claim 1.

19. A non-transitory computer-readable medium storing a computer program according to claim 18.
